(19) United States
(12) Patent Application Publication
(10) Pub. No.: US 2014/0147467 A1
(43) Pub. Date: May 29, 2014

(54) Novel broth medium and blood free solid media

(71) Applicants: Alejandro Javier Yanez Carcamo, Valdivia (CL); Juan Guillermo Carcamo, Valdivia (CL); Victor Hugo Olavarria, Valdivia (CL)

(72) Inventors: Alejandro Javier Yanez Carcamo, Valdivia (CL); Juan Guillermo Carcamo, Valdivia (CL); Victor Hugo Olavarria, Valdivia (CL)

(21) Appl. No.: 13/694,364

(22) Filed: Nov. 26, 2012

(51) Int. Cl. C12N 1/20; C12R 1/01

(52) U.S. Cl. CPC C12N 1/20; C12R 1/01
USPC 424/234.1; 435/253.6; 530/350

(57) ABSTRACT

The present invention relates to new contamination resistant artificial media for the routine cultivation of P. salmonis. In particular, it discloses first a liquid media based named Austral-SRS Broth that extensively require iron and sodium chloride; and, second, a blood-free agar media comprising tryptone soy with either ferric or ferrous salts (Austral-TSFe) agar or the use of hemoglobin (Austral-TSHem) as a source of iron in the agar. Also disclosed is a method for their preparation as well as their application in the development of antigens and low cost vaccines for protection against Piscirickettsiosis and the use of the medias for a kit that evaluate the antibiotic resistance appearance in different strain of P. salmonis isolated from the salmon industry.

### FIELD OF THE INVENTION

The present invention relates to new contamination resistant artificial media for the routine cultivation of Piscirickettsia salmonis and bacteria's from the Piscirickettsia genus. In particular, it discloses first a blood-free liquid media based on austral-SRS Broth; and, second, agar media comprising tryptone soy with either ferric/ferrous salts (TSFe) agar or hemoglobin (TSHem) as a source of iron and the use of sodium chloride as essentials materials for the grow of the bacteria. Also disclosed is a method for their preparation as well as their application in the development of low cost vaccines for protection against Piscirickettsiosis and for a validated method for evaluation of the appearance of antibiotic resistance.

### BACKGROUND OF THE INVENTION

Piscirickettsia salmonis is the first Gram-negative, intracellular bacterial pathogen isolated from fish and constitutes one of the main problems in farmed salmonids and marine fish (see review Almendras & Fuentealba 1997, Mauel & Miller 2002, Fryer & Hedrick 2003). Despite that the outbreaks of the disease occurred late 1980s; the microbial agent was unknown until the end of the 1990s, when Fryer, Lannan, Garces, Larenas & Smith (1990) reported that P. salmonis could be isolated in vitro conditions using Chinook salmon embryo (CHSE-214) cell line. Since then, different types of cell lines have been routinely used to culture P. salmonis, being considered the antibiotic-free CHSE cell line as the accepted diagnostic “gold standard”. However, contamination of the cells is one of the major problems when inoculated with the tissues collected from morbid fish; therefore, the use of artificial media is an alternative that shows potential and would relieve facilities of the cost of maintaining cell lines and eliminates heavily contaminated host cell debris.

The use of an enriched sheep blood agar prepared with cysteine, has been proposed for laboratory culture and study of some physiological characteristics of P. salmonis strains (Mauel, Ware & Smith, 2008). Studies by, Mikalsen, Skjaervik, Wiik-Nielsen, Wasmuth & Colquhoun (2008), have described another agar culture medium based on Cysteine Heart Agar supplemented with 5% ovine blood (CHAB), which was tested in field conditions to evaluate its efficacy for the recovery of P. salmonis from an outbreak in Atlantic salmon (Salmo salar) in Norway as well as to study the phenotypic and genetic characterization of this microorganism. A major disadvantage of the blood supplement was its focus of contamination as well as the difficulty of obtaining it in some countries. Moreover, Piscirickettsia salmonis can be efficiently grown in established insect and fish tissue culture cells, yielding up to 100 times in Sf21 cells than CHSE-214 cells. However, contamination of the cell cultures debris is one of the major problems (Birkbeck T. et al, 2004). Therefore, the use of artificial medias which are blood free, cell free and extracellular free component is an alternative that shows high potential and would relieve facilities of the cost of maintaining cell lines and eliminates the problem of heavily contaminated host cell debris and represent a new alternative to grow bacterial of the Piscirickettsia genus. Also, the media showed to have different uses: preparation and purification of antigens for low cost vaccines

### SUMMARY THE INVENTION

The present invention provides for the first time two alternative agar media suitable for use in the routine culture of P. salmonis in the absence of enriched blood and one liquid media.

In one embodiment, the present invention provides a blood-free agar media comprising tryptone soy with hemoglobin (TSHem) agar for use in the culture of P. salmonis; and, in another embodiment, the present invention provides a blood-free media comprising tryptone soy with ferric nitrate (TSFe) agar for use in the culture of P. salmonis.

In one embodiment, the present invention provides a blood-free liquid media comprising AUSTRAL SRS Broth for use in the culture of P. salmonis whereupon bacteria in a period of from 5 to 10 days.

In another embodiment the present invention provides a culture media comprising tryptone soy with hemoglobin (TSHem) agar whereupon P. salmonis grows in a period of from 8 to 10 days.

In another embodiment the present invention provides a validated kit for the evaluation of appearance of antibiotic resistance.

In another embodiment the present invention provides a method of preparing a blood-free media (liquid and solid) for use in the culture of P. salmonis comprising tryptone soy agar wherein said method comprises the step of adding a source of iron to said media.

In another embodiment the present invention provides a method of preparing a blood-free media (liquid and solid) for use in the culture of P. salmonis comprising tryptone soy agar wherein said method comprises the step of adding a source of sodium chloride to said media.

In yet another embodiment the present invention provides a method of preparing a blood-free media for use in the culture of P. salmonis comprising tryptone soy agar wherein said method comprises the step of adding a source of iron to said media wherein said source of iron is comprised of either ferric nitrate (or any salts of iron, such as chloride, citrate, sulfate, etc.) or hemoglobin or other chemical state like ferrous state.

In yet another embodiment the present invention provides a method for providing a suitable platform to simplify the preparation of P. salmonis cells for genetic-and-serological studies wherein said method comprises the purification of total protein and membrane protein obtained from P. salmonis grown in absence of blood and extracellular component to be used for ELISA and Vaccines uses (FIG. 8).

It will be understood by those skilled in the art that the term blood-free as used herein refers to the absence of enriched blood.

Iron is an essential element for most bacteria, serving as a cofactor in key metabolic processes such as nucleotide biosynthesis, electron transfer, and energy transduction. Most bacterial pathogens require iron (in minimal amount) for growth and to establish an infection, and thus they have developed efficient mechanisms to obtain iron from the host (Ratledge & Dover 2000).

The present invention demonstrates for the first time that iron and sodium chloride are the raw material that P. salmonis needs to grow.

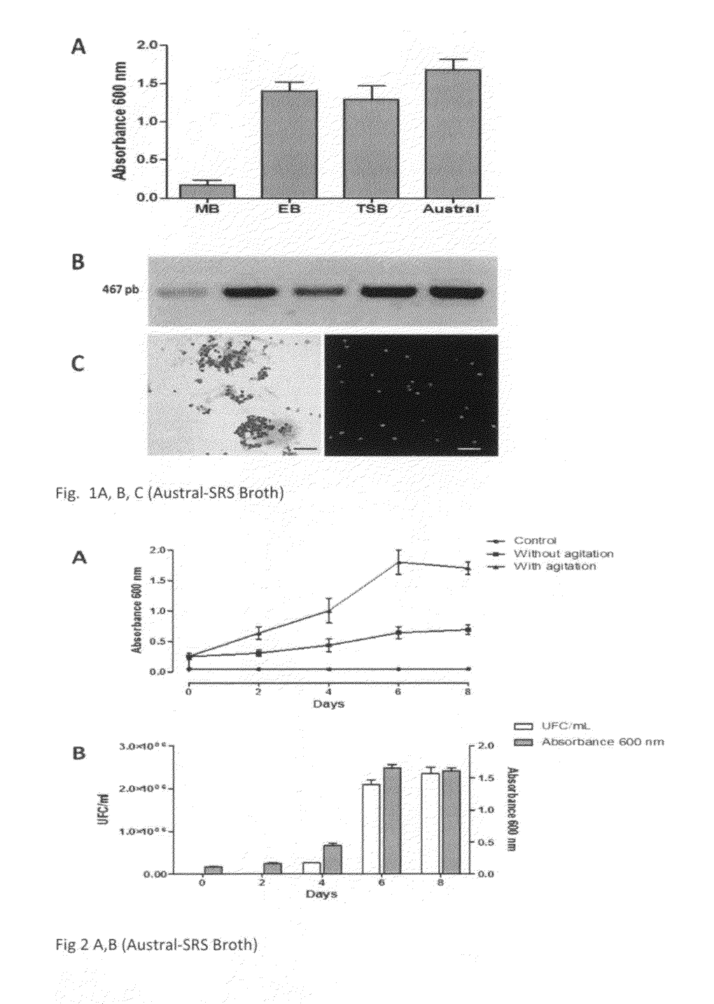

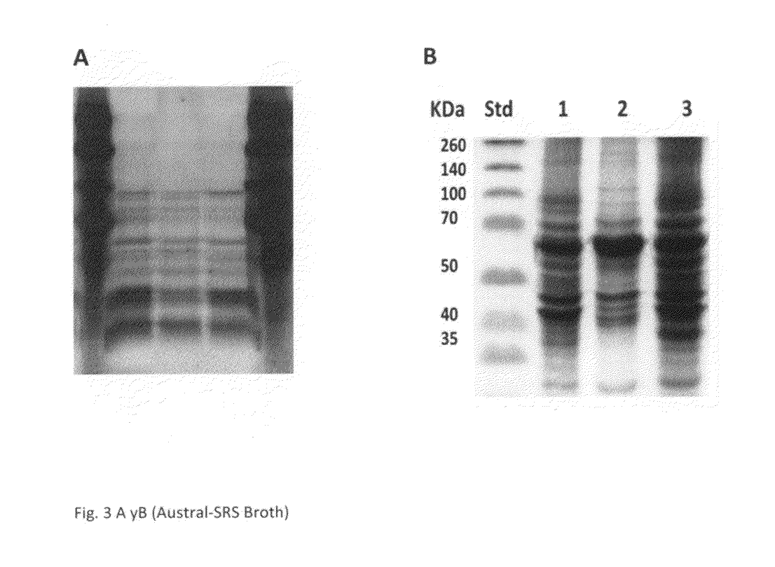

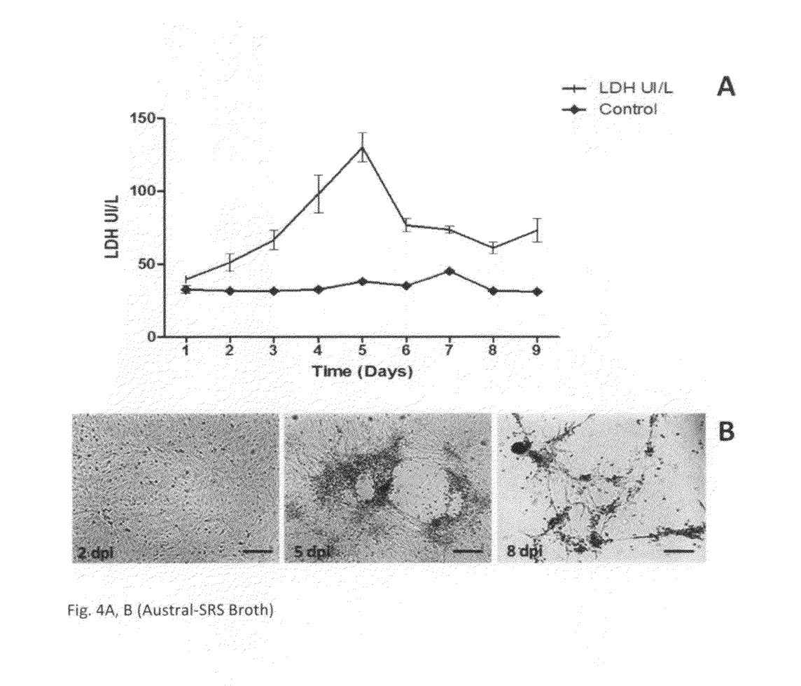

In order to examine the properties of a marine-based broth supplemented with L-cysteine named AUSTRAL-SRS Broth P. salmonis strains were growth which reached approximately 1.8 by measuring the absorbance at 600 nm in six days at 18° C. (FIG. 1A). PCR and IFAT were used to confirm that P. salmonis is able to grow (FIG. 1B,C). Maximum cell density was obtained with agitation (50 rpm); with 64.7% more than the density obtained in the static Erlenmeyer flasks after 6 days of incubation (FIG. 2A,B). The bacterial count showed that the number of cultivable bacteria during the first 5 days decreased by 4 log-units from an initial inoculum of 10⁷ CFU ml⁻¹ (equivalent to 10⁸ cells ml⁻¹). After this period, the number of culturable bacteria increased 2 log-unit of CFU ml⁻¹ at the end of the experiment, allowing the detection of 10⁵ P. salmonis CFU ml⁻¹ (FIG. 2B). Interestingly, several passages (n=6) did not alter the culture kinetics. Moreover, we report for the first time the purification of DNA, LPS and total or membrane protein obtained from P. salmonis grown in this liquid medium, providing a suitable platform to simplify the preparation of P. salmonis cells for genetic-and-serological studies (FIG. 3A, B). Moreover, the results of the cytopathic effect test showed that P. salmonis grown in Austral-SRS Broth maintain their virulence properties inducing the apoptosis after 3 days (FIG. 4) and makes this medium a good candidate for its successful growth and is a first-rate material for the development of low cost vaccines and new method for antibiotic resistance.

The analysis of protein profile between Austral-05 strain and the LF89 strain showed that both strain have a different proteome. Also the CPE of was accompanied by a significant increase until day 5 in the level of LDH liberation in SHK-1 cells inoculated with P. salmonis Austral-05 strain whereas LF89 showed an increased LDH liberation after 10 days (data Not shown). The greatest level of liberation of PLH is produced by greater damage of the cell membrane (FIG. 4A).

In order to examine if P. salmonis can suffer a reduction in growth, the type strain was subjected to three serial passages using as inoculum for each passage a loop of 10 μl volumes obtained from the preceding bacterial culture after 10 days of incubation. Moreover, we analyzed the membrane proteins profiles due to the possible changes in the component of the bacterial protein as described by Pot, Vandamme & Kersters (1994) and also by sodium dodecyl sulfate-polyacrylamide gel electrophoresis (SDS-PAGE; Laemmli 1970).

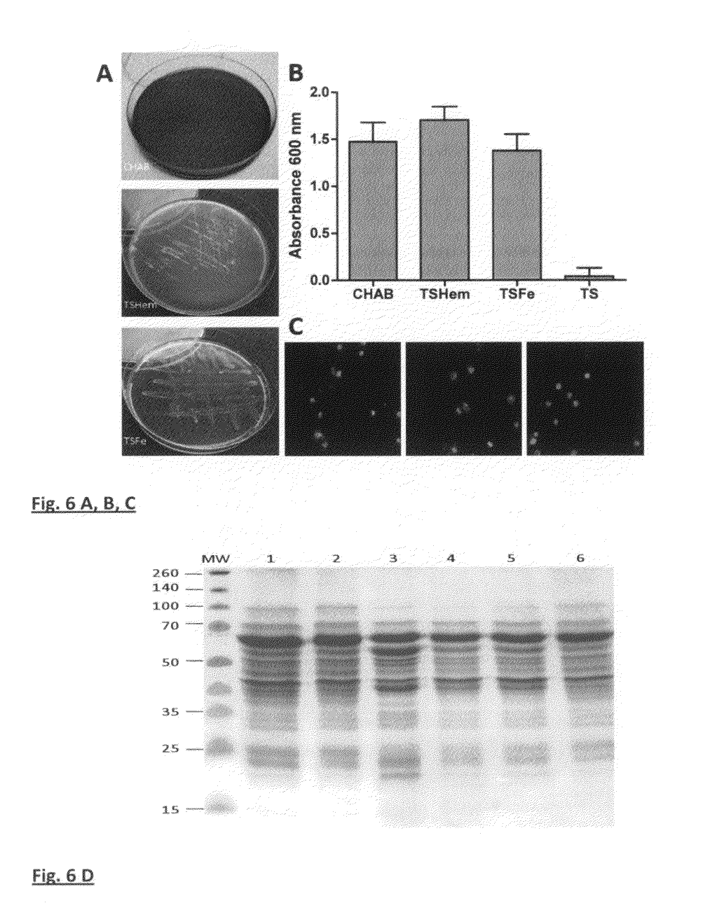

Subculture on TSFe and TSHem did not alter the growth kinetics, regardless of the number of passages, although no individual P. salmonis colonies were observed, after two passages the bacterial strains adapted to the media a started to grow faster (data not shown). With regard to the analysis of whole cell envelope proteins, regardless of the subculture, all P. salmonis strains presented a similar profile, displaying a considerable number of common bands between 116 and 25 kDa (FIG. 6B). Similar results have been reported by Barnes, Landolt, Powell & Winton (1998), who noted that the P. salmonis strains contained a large apparently similar protein, with five major protein bands with molecular masses of 30, 42, 58, 70 and 105 kDa in protein extracts. Three extracts of P. salmonis were performed in order to evaluate the possibility to use as a vaccine (P1, P2 and P3). Only the antigen preparation of P1 showed the ability to largely protect salmon (15% of mortality) against an challenge that induce an 90% of mortality in non-vaccinated fish. Thus, the present invention provides a method for providing a suitable platform to simplify the preparation of P. salmonis cells for genetic-and-serological studies wherein said method comprises the purification of total protein and membrane protein obtained from P. salmonis grown in absence of blood and extracellular component to be used for ELISA and Vaccines uses (FIG. 8).

In yet another embodiment the present invention provides a method for the use of the Austral-SRS broth for the evaluation of minimal inhibitory concentration (MICs) of antibiotics or others drugs for the P. salmonis strains. The Austral-SRS broth was used following a broth micro dilution method with the incubation time, and temperature required for P. salmonis (Yañez et al., 2012a).

### BRIEF DESCRIPTION OF THE DRAWINGS

This patent application contains at least one drawing executed in color. Copies of this patent application publication with color drawing(s) will be provided by the Office upon request and payment of the necessary fee.

### Figure Legend

FIG. 1. Comparison of the growth of Piscirickettsia salmonis type strain in two of the 110 distinct marine- or tryptone soy-broth formulations tested in this study. TSFe broth corresponds to tryptone soy supplemented with ferric nitrate. Growth tests performed with P. salmonis on the remaining 108 versions media did not support the growth of the type strain after 10 days of incubation at 18° C.

FIG. 2. Average growth of all Piscirickettsia salmonis strains used in this study in AUSTRAL-SRS Broth with or without agitation (A) and the concentration of viable bacteria reached with agitation (B). The control is liquid culture without the addition of bacteria and CFU, colony forming unit. The vertical lines show standard deviation.

FIG. 3. SDS-PAGE of (A) silver-stains of lipopolysaccharides and (B) Coomassie stains of whole cell envelope proteins from Chilean Piscirickettsia salmonis isolates and the type strain cultivated in AUSTRAL-SRS Broth. Lanes: MW, molecular size markers; 1, LF-89^{T}; 2, PPT-005 and 3, PPT-0015. Numbers on the right indicate the positions of molecular size markers (kDa).

FIG. 4. LDH liberation (A) and the cytopathic effect (B) in SHK-1 cells infected with Piscirickettsia salmonis. Negative control corresponds to SHK-1 non-infected with the pathogen.

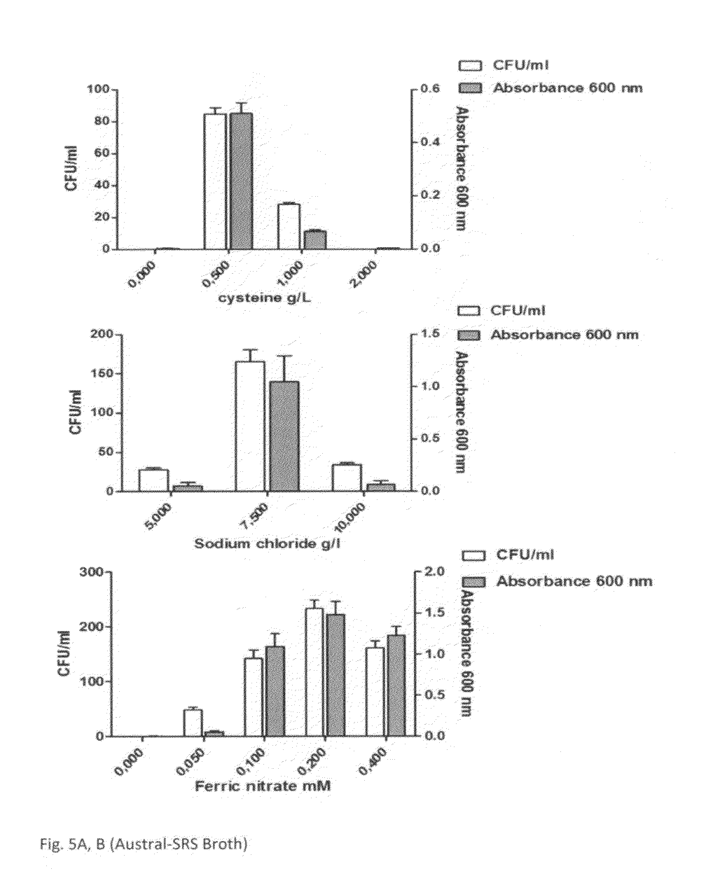

FIG. 5. Illustrates the optimal concentration of L-cysteine, sodium chloride and ferric nitrate used in the two novel blood-free solid media for the culture of the P. salmonis.

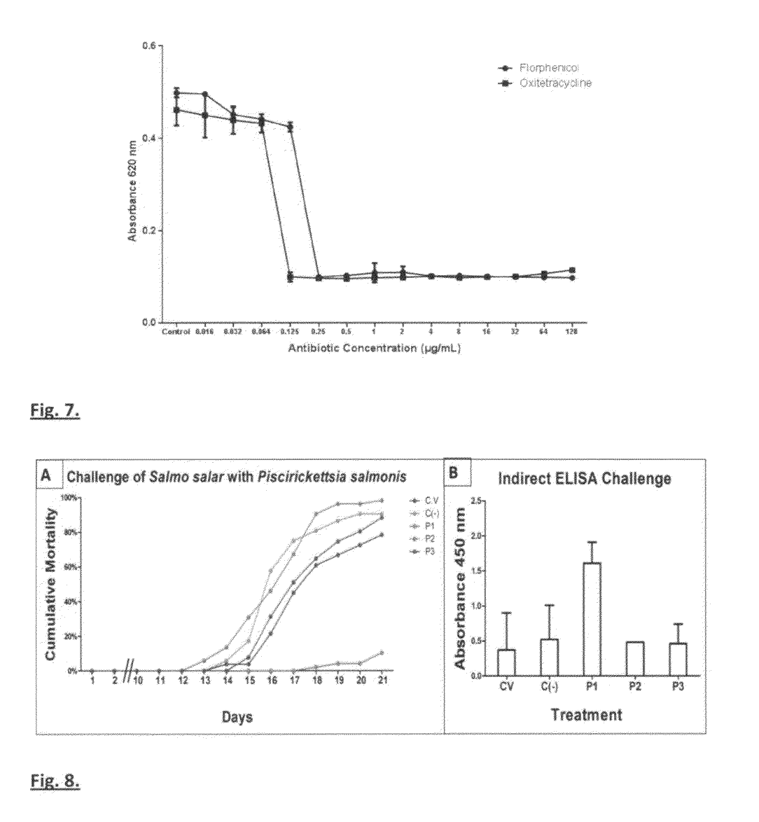

FIG. 6A-D. Show Colonies (A), growth (B) and detection of the P. salmonis strain PPT-05 using indirect immunofluorescence microscopy (C) obtained after 6 or 10 days of incubation at 18° C. cultured on the three CHAB, TSHem and TSFe agar. Vertical lines represent standard deviation. (D) FIGS. 7A and B. A) Show a comparison of whole cell envelope proteins obtained from type strain LF-89^{T} cultured in different agar media using SDS-PAGE gel electrophoresis stained with Coomassie blue R. Lanes: MW, molecular ruler (Spectra™ Multicolor Broad Range Protein Ladder, Fermentas Life Science); 1, CHAB; 2, TSHem and 3, TSFe agar. Lanes: 1 to 3, initial passage and 4 to 6, profile of the type strain after the first (4), second (5) and third (6) passage onto TSFe agar. Numbers on left indicate the positions of molecular size markers (kDa).

FIG. 7. Illustrate the results of the grow of the strain Autral-005 of P. salmonis in Austral-SRS broth in the presence of different concentration of florfenicol and oxytetracycline, in order to evaluate the minimum inhibitory in in vitro conditions for these antibiotics. The results reveal that this strain have different MIC for oxytetracycline (0.124 ugml⁻¹) and florfenicol (0.5 ug/ml⁻¹).

FIG. 8. Illustrate the challenge of salmo salar vaccinated with three prototipes of antigen preparation obtained with P. salmonis grow in Austral-SRS broth; P1, P2 and P3. More over the salmon were vaccinated with adjuvant (C−) and commercial vaccines (C+). The results reveals that the P1 vaccine prototype was able to induce an 90% of protection against the challenge with p. salmonis. B) The antigens were used to prepare an ELISA in order to evaluate the induction of specific antibodies in the salmon. Only P1 induce a large induction of antibodies immune response.

### DETAILS OF THE INVENTION

### Solid Media:

Before the solid medium was used to culture bacteria from the Piscirickettsia genus (P. salmonis), various marine or trytone soy compositions and growth conditions were screened by inoculating the P. salmonis type strain directly onto each agar medium. A total of two versions of trypticase soy agar, designated as Austral-TSFe and Austral-TSHem agar were selected as ideal media to propagate and culture P. salmonis. The first medium contains 45 g tryptone soy agar, 5 g D-glucose, 7.5 g sodium chloride (NaCl) and 0.2 mM ferric nitrate, components that are dissolved by agitation in 1000 ml of distilled water and sterilized by autoclaving (121° C., 15 min). Then, the solution is cooled to 55° C. and 5% fetal bovine serum (FBS) and 0.5% L-cysteine are aseptically added. The TSHem agar has the same components that TSFe medium, except 0.05 mM ferric nitrate, being replaced by the addition of 50 ml BD BBL™ Hemoglobin Solution 2% after sterilization. To determine the optimal concentration of some components (i.e. L-cysteine, NaCl and ferric nitrate) an independent study with each component was preliminary tested (FIG. 5). In addition, plates of CHAB (Mikalsen et al. 2008) were included for comparative purposes and negative controls corresponded to each medium without the bacterium, which were processed in the same manner as described above. The experiment was carried out in six replicates for each medium. In this study, P. salmonis isolate Austral-05 recovered in 2008 from diseased farmed rainbow trout (Oncorhynchus mykiss) in Chile and the type strain P. salmonis ATCC VR-1361 (equivalent to LF-89) from the American Type Culture Collection and originally isolated from Coho salmon (Oncorhynchus kisutch) were used. Both bacterial strains were confirmed as P. salmonis using PCR-based analysis described by Mauel, Giovannoni & Fryer (1996) and also by an indirect fluorescent antibody test (IFAT, SRS-BiosChile) according to the manufacturer's recommendation (FIG. 1). To compare recovery, identical portions (0.1 ml) of freshly thawed material from frozen CHSE-214 cell culture supernatants, infected with each P. salmonis strain was spread over the surface of both Austral-TSFe and Austral-TSHem agar and the P. salmonis biomass was recorded after incubation at 18° C. for 15 days. Briefly, bacterial colonies were collected from each agar plate, suspended in 1 ml of L-15 Leibovitz medium containing 10% of FBS and absorbance at 600 nm values were recorded in order to quantify the produced biomass.

The growth of P. salmonis onto the two blood-free agars takes from 8 to 10 days for visible pin-point colonies to appear, which were identical to those reported previously by Mikalsen et al. (2008), but in colour are white (FIG. 6A). Although between the three media tested, no significant difference (P<0.05) in bacterial growth was observed. Of the three solid media, P. salmonis showed the best growth onto the Austral-TSHem plates with an average in absorbance of about 20% and 25% more than CHAB and Austral-TSFe, respectively (FIG. 6B). These findings can be attributed to qualitative differences in the composition of the media, mainly in the iron source. As expected, the bacterial colonies were not recovered onto plates used as negative control as well as those plates prepared without cysteine (data not shown). The importance of the addition of cysteine for the growth of P. salmonis was also found by Mauel et al. (2008).

Even though the growth on CHAB media takes from 5 to 6 days for visible colonies to appear, the two versions of trypticase soy agar have the advantage that do not require enriched blood. This indicates that the replacement of blood by the addition of ferric nitrate or BD BBL™ Hemoglobin Solution is a better alternative for the growth of P. salmonis. Moreover, the results indicate that the bacterium does not grow on plates without the iron source.

To confirm that P. salmonis was the organism growing, at the end of incubation, a single colony was picked from each agar plate and suspended in 100 μl to standard microscopical examination, as well as analysis by IFAT and PCR (FIG. 6C). Microscopic observations under phase contrast of P. salmonis cultured onto all media did not demonstrate changes in morphology and size, appearing as Gram-negative cocci by Gram staining. All samples were positive by indirect immunofluorescence assays and PCR analysis, confirming the cultured organism's identity as P. salmonis (FIG. 6C). Also, the p. salmonis grow in these media showed to have the ability to induce the symptoms of the picirickettsiosis in a range of 15 to 30 day after a challenge of salmon (three farming salmon) and induce the death of salmon. A lethal study studied reveals that P. salmonis grow in the solid and liquid media were able to induce 90% of mortality with an intraperitoneal injection.

In summary, we concluded that these new blood-free agar media are suitable to be use in laboratory for the routinely culture of P. salmonis, being Austral-TSHem medium the most appropriate for giving the highest number of cells per plate of this specie. Moreover, the purification of the membrane protein obtained from P. salmonis grown in absence of blood, provides a suitable platform to simplify the preparation of P. salmonis cells for genetic-and-serological studies. Moreover, Austral-TSFe or Austral-TSHem medium should facilitate the in vitro drug susceptibility testing of this fastidious pathogen and also the preparation of P. Dissolve the 1-4 ingredients in 784 ml of distilled water and sterilize by autoclaving. Allow to cool to room temperature to 50° C. and aseptically add the ingredient 5 to 8. The cysteine solution was prepared by dissolving 10 g of L-cysteine in 100 ml deionized water. Filter sterilize (0.2 μm filter) and store at 4° C.

Austral-SRS Broth media

Before the liquid medium was used to culture all P. salmonis, a total of 110 distinct broth formulations were screened by inoculating the P. salmonis type strain directly into each liquid medium. Two of the 110 formulations tested showed growth at different levels and only the medium named AUSTRAL-SRS Broth, generated a the better growth compared with the other media (FIG. 1), while the remaining 108 formulations did not support the growth of this strain.

In each medium, the P. salmonis type strain was identified by PCR analysis and IFAT tests and confirmed by partial ITS and 16S rRNA gene sequencing. Thus, AUSTRAL-SRS Broth was selected as ideal candidate for further studies. According to the criteria described by McGann et al. (2010) a liquid medium must induce rapid, high-density bacterial growth as measured by optical density at 600 nm and promote the efficient growth of low bacterial inoculums.

When the growth curves of all P. salmonis were determined by measuring absorbance, the three strains grew well without and with a moderate agitation at temperature between 10 and 20° C. (FIG. 2A). However, statistical analysis revealed significant difference (P<0.05) in the growth of all P. salmonis that were dependent on the movement of the broth culture. Maximum cell density was obtained with agitation (50 rpm); with 64.7% more than the density obtained in the static Erlenmeyer flasks after 6 days of incubation and with better performance at 18° C. In fact, the growth of P. salmonis with gently shaking was characterized by a short lag phase (approximately 18 h) followed by a rapid logarithmic growth, achieving a maximal optical density of approximately 1.8 after 6 days incubation. From this point, the growth remained nearly constant during the experimental period (10 days) at values of approximately 1.68. However, the media allow to gown the bacteria without agitation.

It is important to point out that no difference was observed in the growth dynamics between the two P. salmonis isolates and type strain, regardless of the number of replicates and the agitation condition, suggesting that in AUSTRAL-SRS Broth the behavior of this bacterium is reproducible. In fact, when a serial passage experiment was carried out, the P. salmonis type strain displayed similar growth kinetics, regardless of the number of passages, reaching absorbance values between 1.7 to 1.8 (data not shown). Although the P. salmonis strain grew on TSFe agar, poorly defined colonies were produced, leading to inaccuracies in estimations of the CFU concentration. Thus, the bacterial count showed that the number of culturable bacteria during the first 5 days decreased by 4 log-units from an initial inoculum of 10⁷ CFU ml⁻¹ (equivalent to 10⁸ cells ml⁻¹). After this period, the number of culturable bacteria increased 2 log-unit of CFU ml⁻¹ at the end of the experiment, allowing the detection of 10⁵ P. salmonis CFU ml⁻¹ (FIG. 2B).

Taking into consideration that the persistence of culturable cells was lower than the expected in correlation to absorbance values, the quantitative real-time PCR (qRT-PCR) designed by Karatas et al. (2008) was applied (data not shown). A slight increase in number of P. salmonis cells from the initial inoculum was determined during the first 6 days (data not shown). Then, 12 days after the beginning of the experiment, qRT-PCR count showed that the number of bacteria increased by 1 log-unit, reaching 7.3×10⁹ cells. Therefore, P. salmonis lost its ability to grow on solid media, nonetheless, retained viability in liquid media.

Despite that the P. salmonis strains grew on distinct enriched sheep blood agar prepared with cysteine (Mikalsen et al. 2008, Mauel et al. 2008) as well as in the blood-free medium employed in this work, it is known that the efficiency of recovering P. salmonis from all media is not suitable for the determination of the concentration, mainly because bacterial growth is extremely slow. In fact, growth on solid media usually takes from 4 to 8 days for visible colonies to appear (Mikalsen et al. 2008, Mauel et al. 2008). This disadvantage leads us to express the growth of P. salmonis in absorbance units or cells.

Microscopic observations under phase contrast of P. salmonis cultured into AUSTRAL-SRS broth did not demonstrate changes in morphology and size, appearing as Gram-negative cocci. An aliquot of each culture was observed under epifluorescence microscope every day, in order to confirm the presence of P. salmonis when exposed to a commercial rabbit FITC-conjugated anti-P. salmonis antibody, showing a strong and positive specific reaction. As expected, liquid culture without addition of P. salmonis did not yield any growth.

The result of the LPS profiling showed that all Chilean P. salmonis isolates and the type strain displayed a similar LPS pattern with a ladder of low-molecular-weight (LMW) O-antigen bands, but less abundant high-molecular-weight (HMW) species were present (FIG. 3A), matching the typical profile of the P. salmonis species to that reported by Kuzyk et al. (1996). These authors noted that silver staining of PK digested P. salmonis revealed a ladder-like banding pattern of carbohydrates ranging from 16 to 35 kDa in size with a discrete band around 20 kDa and an intensely stained major band around 11 kDa. It is important to note that Kuzyk et al. (1996) using rabbit polyclonal antibodies recognized this band as 11-kDa carbohydrate antigen corresponding to a lipooligosaccharide component of LPS, which was confirmed by other studies (Barnes et al. 1998, Jamett et al. 2001). With regard to the analysis of whole cell envelope proteins, all Chilean P. salmonis strains presented a similar profile, displaying a considerable number of common bands between 116 and 25 kDa (FIG. 3B). Similar results have been reported by Barnes et al. (1998), who noted that P. salmonis studied contained a large apparently similar protein, with five major protein bands with molecular masses of 30, 42, 58, 70 and 105 kDa.

Adherence, invasion and intracellular replication in the host cells are important for pathogenesis by intracellular pathogens (Finlay & Falkow 1997, Nobbs et al. 2009). It is well known that P. salmonis replicates by binary fission within membrane-bound cytoplasmic vacuoles in cells of susceptible fish hosts or fish cell lines inducing a characteristic cytophathic effect (CPE) (Fryer & Hedrick, 2003). Moreover, many studies on P. salmonis consider that the Chinook salmon embryo (CHSE-214) cells offers considerable advantages over other fish, insect and frog tissue culture cells. However, Birkbeck et al. (2004) showed that P. salmonis replicates in higher titers in an insect cell line than in the CHSE-214 cells that is normally used to culture the organism and that P. salmonis retains virulence for Atlantic salmon (Salmo solar) after repeated culture in insect cells. On the other hand, the tissue chosen for the isolation of P. salmonis during active infection in salmonids is kidney and SHK-1 is a cell line from Salmo solar head kidney, which exhibits macrophage properties (Dannevig et al. 1997).

Unpublished work in our laboratory showed that P. salmonis strains produced similar CPE in CHSE-214 cells than SHK-1 cells, although the cell sheet is completely lysed 15 days post-infection. Therefore, we decided to evaluate the effect of P. salmonis cells cultured in AUSTRAL-SRS Broth into the SHK-1 cell line. Microscopic analysis revealed similar infectivity patterns as those reported by Olavarria et al. (2010), observing the CPE after 3 days post-infection and due to their growth conditions, the infection was spread to neighboring cells with total degenerative changes in the SHK-1 cell line after 5 days of incubation (FIG. 4B). In addition, the CPE was accompanied by a significant increase until day 5 in the level of LDH liberation in SHK-1 cells inoculated with P. salmonis. In fact, on the second day of the experiment the P. salmonis studied presented a significant increase in liberation up until day 5 from the onset of the study, when P. salmonis showed the greatest level of liberation owing to greater damage of the cell membrane (FIG. 4A). After this, LDH analyses showed that the values declined very rapidly during the first 24 h, followed by a stabilization around 70 LDH Ul/L until the end of the experiment. As expected for the monolayers non-inoculated with P. salmonis isolates, negative results of the CPE test and LDH analysis were found. Further confirmation of the identity of P. salmonis isolates was provided by IFAT test.

Our results indicate that in addition to having the capacity to establish the infectious cycle and overcome cell barriers, this bacterium may also have a greater replication index and consequent liberation of cells with infective capacity similar to other fish pathogens (Ortega et al. 2011). Therefore, the culture of the bacteria in broth medium does not affect the infective properties in in vitro and in vivo conditions, challenge experiments with fish confirm that the Chilean isolates grow in AUSTRAL-SRS broth do cause the observed disease and the death of salmon.

Austral SRS broth is a supplemented media for Piscirickettsia salmonis culture that is composed in base to Peptone and yeast extract as a source of nitrogen, vitamins and minerals (Table 2). The high sodium chloride content helps to simulate sea water; numerous minerals are also included to duplicate the major mineral composition of sea water. Preparation for 1 L: Suspend the ingredients item 1 to 21 in 800 ml of distilled water and sterilize by autoclaving at 121° C. for 15 minutes. Allow to cool to room temperature and aseptically add the ingredients 22 to 50. According to Smith (1998) the composition of the medium used for susceptibility should give sufficiently good growth conditions for the strains to be tested and must not contain material interfering with the test itself or reacting with any antimicrobial tested. Although the CLSI (2006a; b) frequently suggests that the susceptibility testing (disc diffusion and MIC assays) should be performed using Mueller-Hinton medium or based on some version; until now, P. salmonis has not been included in the guidelines.

Recently the use of an enriched sheep blood agar prepared with cysteine (Mauel et al. 2008) and another agar culture medium based on Cysteine Heart Agar supplemented with 5% ovine blood (Mikalsen et al. 2008) has been proposed for laboratory culture and study of some physiological characteristics of P. salmonis strains. Although both media have also been used for the drug susceptibility testing of this fastidious pathogen, poorly defined zones around the disk are produced, leading to inaccuracies in estimations of the inhibition zone sizes (unpublished data.). Our study was performed using AUSTRAL-SRS broth as the base medium which showed a good overall correspondence with the MICs obtained with a fluorinated structural synthetic analog of thiamphenicol and chloramphenicol, florfenicol (FLO) and oxytetracycline (OTC), two bacteriostatic agents with broad spectrum activity has often been the drugs of choice for treating outbreaks delivered either in food or by injection by Smith et al. (1996). Moreover, considering that P. salmonis replicates by binary fission within membrane-bound cytoplasmic vacuoles in cells of susceptible fish hosts or fish cell lines inducing a characteristic cytopathic effect (Fryer and Hedrick 2003), the data obtained in our laboratory indicates that this medium was successfully used to tests antibiotic susceptibility of P. salmonis isolates (FIG. 7). No differences were observed among the MIC values obtained for each strain in the independent experiments performed (n=10), which included separate preparation of inoculum and/or the test itself. In the same way, MIC values of E. coli ATCC 25922 grown in CAMH broth showed acceptable values for all drugs. The MICs for the P. salmonis strains were determined by following a standardized broth micro dilution method in the Austral-SRS broth with the incubation time, and temperature as required for P. salmonis (Yañez et al., 2012a). The strains were inoculated in AUSTRAL-SRS broth medium which contained 2-fold dilutions of the antibacterial agents tested, ranging from 0.016 to 256 μg ml−1. OTC and FLO used in the MIC testing were all obtained from Sigma-Aldrich. Standard stock solutions were prepared by dissolving 10 mg of each antibacterial agent with 500 μl of methanol 100% (OTC) and ethanol 95% and the final volume adjusted with distilled water to 10 ml. All stock solutions were stored at 4° C. and used within 24 h. Each micro dilution tray included a growth control well (without antibiotics) and a negative (uninoculated) well as well as three controls included the solvents methanol and ethanol in amounts corresponding to the highest quantity present in the assay. Each test was carried out ten times for each strain and the MIC value was defined as the lowest concentration exhibiting no visible bacterial growth at 18° C. for 92 to 96 h and/or 116 to 120 h. In addition, the microtitre plates were read with a spectrophotometer in order to compare any possible difference in sensitivity between both reading methods. As expected, liquid culture without the addition of P. salmonis did not yield any growth. The isolate austral-005 had a low susceptibility and fell within a narrow range. For FLO, the isolates showed MIC values ranging from 0.25 to 0.5 and for OTC between 0.125 to 2.5 μg mL−1. In addition, P. salmonis ATCC VR-1361 showed MIC value of 0.25-0.5 μg mL−1 for OTC and FLO (FIG. 7).

In conclusion, from a microbiological point of view, one of the major constraints on studies of P. salmonis is the ability to isolate different structural component (i.e. DNA, LPS and proteins) free of host cell. We report, for the first time the purification of DNA, LPS and proteins obtained from P. salmonis grown in a liquid medium, providing a suitable platform to simplify the preparation of P. salmonis bacterium for genetic-and-serological studies. Moreover, the results of the cytopathic effect test showed that P. salmonis grown in Austral-SRS Broth maintain the virulence, genome and proteome properties making this medium an excellent media to successful grow large amount of P. salmonis in any industrial systems. Indeed, the Genome sequence analysis is often used to distinguish different strains of a genus group. The internal transcribed spacer which is in between 16S and 23S ribosomal RNAs was characterized showing that the strains grow in solid and liquid medias developed belong to the Piscirickettsia genus. Thus, the embodiment of the medias described related to the grow of P. salmonis, wherein said bacterium fitting to the Piscirickettsia genus is characterized to have and ITS sequence which is at least 96% to the sequence:

5′TATTTAATTAACGAGTCTTGGTAATTTTTGAAAACCGGTGTTGAGATA
TAATTTTGATTGGTTTTAGTTAATAGATTATAGATTTATTGATATAAGAC
TT3′.

### REFERENCES

Almendras F. E. & Fuentealba I. C. (1997) Salmonid rickettsial septicemia caused by Piscirickettsia salmonis: a review. Diseases of Aquatic Organisms 29, 137-144.
Barnes M. N., Landolt M. L., Powell D. B. & Winton J. R. (1998) Purification of Piscirickettsia salmonis and partial characterization of antigens. Diseases of Aquatic Organisms 33, 33-41.
Birkbeck, T., Griffen, A., Reid, H., Laidler, L. and Wadsworth S. Growth of Piscirickettsia salmonis to High Titers in Insect Tissue Culture Cells”. Infection and immunity, June 2004, p. 3693-3694.
Fryer J. L. & Hedrick R. P. (2003) Piscirickettsia salmonis: a Gram-negative intracellular bacterial pathogen of fish. Journal of Fish Diseases 26, 251-262.
Fryer J. L., Lannan C. N., Garces L. H., Larenas J. J. & Smith P. A. (1990) Isolation of a rickettsiales-like organism from diseased Coho salmon (Oncorhynchus kisutch) in Chile. Fish Pathology 25, 107-114.
Laemmli U. K. (1970) Cleavage of structural proteins during the assembly of the head of bacteriophage T4. Nature 227, 680-685.
Mauel M. J. & Miller D. L. (2002) Piscirickettsiosis and piscirickettsiosis-like infections in fish: a review. Veterinary Microbiology 87, 279-289.
Mauel M. J., Giovannoni S. J. & Fryer J. L. (1996) Development of polymerase chain reaction assays for detection, identification, and differentiation of Piscirickettsia salmonis. Diseases of Aquatic Organisms 26, 189-195.
Mauel M. J., Ware C. & Smith P. A. (2008) Culture of Piscirickettsia salmonis on enriched blood agar. Journal of Veterinary Diagnostic Investigation 20, 213-214.
Mikalsen J., Skjaervik O., Wiik-Nielsen J., Wasmuth M. A. & Colquhoun D. J. (2008) Agar culture of Piscirickettsia salmonis, a serious pathogen of farmed salmonid and marine fish. FEMS Microbiology Letters 278, 43-47.
McGann P, Rozak D A, Nikolich M P, Bowden R A, Lindler L E, Wolcott M J, Lathigra R (2010) A novel brain heart infusion broth supports the study of common Francisella tularensis serotypes. J Microbiol Methods 80:164-171
Pot B., Vandamme P. & Kersters K (1994) Analysis of electrophoretic whole-organism protein fingerprints. In: Goodfellow M, O'Donnell A G (eds) Chemical Methods in Prokaryotic Systematics, Chichester, Wiley, p 493-521.
Ratledge C. & Dover L. G. (2000). Iron metabolism in pathogenic bacteria. Annual Review of Microbiology 54, 881-941.
Yáñez, A. J., Valenzuela, K., Silva, H., Retamales, J., Romero, A., Enriquez, R., Figueroa, J., Claude, A., Gonzalez, J., Avendaño-Herrera, R., Carcamo, J, G,. 2012. Broth médium for the successful culture of the fish pathogen P. salmonis. Diseases of Aquatic Organisms 97, 197-205.

TABLE 1
Composition and preparation of 1 liter
of Austral-TSFe and Austral TSHe agar
Constituent

| | | g/L |
|---|---|---|
| Austral-TSFe Agar | | |
| 1. | Pancreatic Digest of Casein | 15 |
| 2. | Papaic Digest of Soybean | 5 |
| 3. | Sodium Chloride | 12.5 |
| 4. | Agar | 15 |
| 5. | Glucose | 5 |
| 6. | Ferric Nitrate | 0.05 |
| 7. | Cysteine | 1 |
| 8. | FBS | 50 ml |
| Austral-TSHem Agar | | |
| 1. | Pancreatic Digest of Casein | 15 |
| 2. | Papaic Digest of Soybean | 5 |
| 3. | Sodium Chloride | 12.5 |
| 4. | Agar | 15 |
| 5. | Glucosa | 5 |
| 6. | Hemoglobina | 1 |
| 7. | Cysteine | 1 |
| 8. | FBS | 50 ml |

TABLE 2
Composition and preparation of AustralSRS-broth

| Amino acids | mg/L |
|---|---|
| Glycine | 0.8 |
| L-Alanine | 0.9 |
| L-Arginine | 12.6 |
| L-Asparagine-H2O | 1.3 |
| L-Aspartic acid | 1.3 |
| L-Cystine 2HCl | 1703.1 |
| L-Glutamic Acid | 1.5 |
| L-Histidine hydrochloride-H2O | 4.2 |
| L-Isoleucine | 5.2 |
| L-Leucine | 5.2 |
| L-Lysine | 7.3 |
| L-Methionine | 1.5 |
| L-Phenylalanine | 3.2 |
| L-Proline | 1.2 |
| L-Serine | 1.1 |
| L-Threonine | 4.8 |
| L-Tryptophan | 1.0 |
| L-Tyrosine disodium salt dihydrate | 5.2 |
| L-Valine | 4.6 |
| Vitamins | 0.0 |
| Choline chloride | 0.1 |
| D-Calcium pantothenate | 0.1 |
| Folic Acid | 0.1 |
| Niacinamide | 0.1 |
| Pyridoxal hydrochloride | 0.1 |
| Riboflavin | 0.0 |
| Thiamine hydrochloride | 0.1 |
| i-Inositol | 0.2 |
| Inorganic Salts | 0.0 |
| Ammonium Nitrate | 0.2 |
| Boric Acid | 2.8 |
| Calcium Chloride (CaCl2) (anhyd.) | 246.1 |
| Disodium Phosphate | 1.0 |
| Ferric Citrate | 12.6 |
| Feric Nitrate | 5.0 |
| Magnesium Chloride | 741.2 |
| Magnesium Sulfate (MgSO4) (anhyd.) | 416.8 |
| Potassium Bromide | 10.1 |
| Potassium Chloride (KCl) | 109.1 |
| Sodium Bicarbonate (NaHCO3) | 240.1 |
| Sodium Chloride (NaCl) | 7123.5 |
| Sodium Fluoride | 0.3 |
| Sodium Phosphate monobasic (NaH2PO4—H2O) | 14.0 |
| Sodium Silicate | 0.5 |
| Sodium Sulfite | 200.0 |
| Strontium Chloride | 4.3 |
| Other Components | 0.0 |
| D-Glucose (Dextrose) | 15600.0 |
| Proteose Peptone No. 3 | 8128.1 |
| Pancreatic Digest of Casein | 7500.0 |
| Soy Peptone | 5000.0 |
| Yeast Extract | 325.6 |
| Fetal bovine serum | 100 ml |

1. A solid and liquid blood-free medium for the cultivation of P. salmonis comprising tryptone soy agar combined with hemoglobin (Austral-TSHem) in a range of 0.2-5.0 g/l and sodium chloride in a range of 3-15 g/l.

2. A solid blood-free medium for the cultivation of P. salmonis comprising tryptone soy agar combined with iron salts in a ferric nitrate or other ferric or ferrous salts (Austral-TSFe) in a range of 5-50 mg/l and sodium chloride in a range of 3-15 g/l.

3. A liquid-free medium (Austral SRS broth) for the cultivation of P. salmonis comprising a complex mixture of material (Table 2) combined with iron salts in a ferric nitrate or other ferric or ferrous salts in a range of 5-50 mg/l and sodium chloride in a range of 3-15 g/l.

4. The solid and liquid blood-free medium of claim 1 or claim 2 wherein said P. salmonis grows in a period of from 8 to 10 days.

5. A method of preparing a blood-free media for use in the culture of P. salmonis according to claim 1 wherein said method comprises the step of adding a source of iron and sodium chloride to said media.

6. A method of preparing a blood-free media for use in the culture of P. salmonis according to claim 2 wherein said method comprises the step of adding a source of iron salts and sodium chloride to said media.

7. A method of preparing a blood-free media comprising Austral-TSHem agar for use in the culture of P. salmonis according to claim 4 wherein said source of iron is hemoglobin.

8. A method of preparing a blood-free media comprising Austral-TSFe agar for use in the culture of P. salmonis according to claim 5 wherein said source of iron is ferric salt or other ferrous salt such as nitrate, chloride sulfate, etc.

8. A method for providing a suitable platform to simplify the isolation of P. salmonis cells for genetic-and-serological studies wherein said method comprises the step of purifying the whole protein, cytosolic and membrane protein for vaccine preparation or ELISA are obtained from P. salmonis grown in absence of blood and cellular component.

9. A blood-free liquid media comprising AUSTRAL SRS broth for use in the culture of P. salmonis.

10. A method comprising AUSTRAL SRS broth, TSHem and TSFe for use in their evaluation of the appearance of antibiotic resistance of P. salmonis.

11. A method of preparing an Austral-SRS broth liquid media for use in the culture of P. salmonis according to claim 9 wherein said method comprises the step of adding a source of iron to said media.

12. A vaccine based in isolated protein from P. Salmonis in the developed methods according claims 5, 6, 7, 8, 9, 10 wherein said method comprise the step of isolation of highly purify P. salmonis in austral SRS broth or solid media


SEQUENCE LISTING (1 sequence)

SEQ ID NO: 1 - DNA, 100 bp, Piscirickettsia salmonis
  misc_RNA (1)..(100)
  ref <308>: DATABASE ACCESSION NUMBER: AY498629
  ref <309>: DATABASE ENTRY DATE: 2004-07-13
  ref <313>: RELEVANT RESIDUES IN SEQ ID NO: (149)..(248)
  ref <308>: DATABASE ACCESSION NUMBER: EU170252
  ref <309>: DATABASE ENTRY DATE: 2007-11-18
  ref <313>: RELEVANT RESIDUES IN SEQ ID NO: (174)..(273)
  ref <308>: DATABASE ACCESSION NUMBER: AF205384
  ref <309>: DATABASE ENTRY DATE: 2000-07-28
  ref <313>: RELEVANT RESIDUES IN SEQ ID NO: (523)..(622)
tatttaattaacgagtcttggtaatttttgaaaaccggtgttgagatataattttgattggttttagttaatagattatagatttattgatataagactt